(19) United States
(12) Patent Application Publication
(10) Pub. No.: US 2003/0097042 A1
(43) Pub. Date: May 22, 2003

(54) Endoscopic system

(76) Inventor: Teruo Eino, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/283,281

(22) Filed: Oct. 30, 2002

(30) Foreign Application Priority Data
Oct. 31, 2001 (JP) 2001-335036

(51) Int. Cl. A61B 1/04

(52) U.S. Cl. 600/118; 600/101

(57) ABSTRACT

Briefly, an endoscopic system in accordance with the present invention consists mainly of: an endoscope to be used to perform endoscopic examination; a first personal computer having a first display unit on which an endoscopic image produced by the endoscope and a control panel image through which the endoscope is controlled or operated are displayed; and a second personal computer connected to the first personal computer over a communications network. The same endoscopic image and control panel image as those displayed on the first display unit are displayed on a second display unit included in the second personal computer. The endoscope can be controlled or operated through the control panel image displayed on the second display unit.

This application claims the benefit of Japanese Application No. 2001-335036 filed in Japan on Oct. 31, 2001, the contents of which are incorporated by reference.

### BACKGROUND OF THE INVENTION

1. Field of the Invention

The present invention relates to an endoscopic system suitable for remotely controlling examination or the like.

2. Description of the Related Art

In recent years, endoscopes have been widely adopted in the fields of medicine and industries alike. Moreover, a technology for performing endoscopic examination by operating an endoscope, to which a controller realized with a personal computer or the like is connected, using the personal-computer has come to attract attention these days.

According to prior arts that relate to endoscopic examination to be performed by operating an endoscope using a personal computer or the like, it is hard and unpractical to operate the endoscope in a place far away from an installation site of the endoscope.

### FEATURE AND SUMMARY OF THE INVENTION

Accordingly, an object of the present invention is to provide an endoscopic system enabling a user to operate an endoscope in a place different from an installation site of the endoscope as if to operate the endoscope at the installation site.

Briefly, an endoscopic system in accordance with the present invention consists mainly of: an endoscope used to perform endoscopic examination; a first personal computer that includes a first display unit on which an endoscopic image produced by the endoscope and a control panel image through which the endoscope is controlled or operated are displayed; and a second personal computer connected to the first personal computer over a communications network. The same endoscopic image and control panel image as those displayed on the first display unit are displayed on a second display unit included in the second personal computer. The endoscope can be controlled or operated through the control panel image displayed on the second display unit.

The above feature of the present invention and the advantages thereof will be further apparent from the following detailed explanation.

### BRIEF DESCRIPTION OF THE DRAWINGS

FIG. 1 shows the overall configuration of an endoscopic system in accordance with a first embodiment of the present invention;

FIG. 2 shows the configuration of an endoscope system included in the endoscopic system of the first embodiment;

FIG. 3 is a block diagram showing the components of a signal processing unit included in the endoscopic system of the first embodiment;

FIG. 4 is a block diagram showing the components of an examination personal computer included in the endoscopic system of the first embodiment;

FIG. 5A shows the monitor screen of the examination personal computer included in the endoscopic system of the first embodiment;

FIG. 5B shows the monitor screen of a center personal computer included in the endoscopic system of the first embodiment;

FIG. 6 shows the overall configuration of an endoscopic system in accordance with a second embodiment of the present invention;

FIG. 7A shows the monitor screen of an examination personal computer included in the endoscopic system of the second embodiment;

FIG. 7B shows the monitor screen of a center personal computer included in the endoscopic system of the second embodiment;

FIG. 8 shows the overall configuration of an endoscopic system in accordance with a third embodiment of the present invention;

FIG. 9A shows the monitor screen of an examination personal computer included in the endoscopic system of the third embodiment;

FIG. 9B shows the monitor screen of a quality control department personal computer included in the endoscopic system of the third embodiment;

FIG. 10 shows the overall configuration of an endoscopic system in accordance with a fourth embodiment of the present invention;

FIG. 11A shows the monitor screen of an examination personal computer included in the endoscopic system of the fourth embodiment;

FIG. 11B shows the monitor screen of a selling agency personal computer included in the endoscopic system of the fourth embodiment;

FIG. 12 shows the overall configuration of an endoscopic system in accordance with a fifth embodiment of the present invention;

FIG. 13A shows the monitor screen of an examination personal computer included in the endoscopic system of the fifth embodiment;

FIG. 13B shows the monitor screen of a repair work personal computer included in the endoscopic system of the fifth embodiment;

FIG. 14 shows the overall configuration of an endoscopic system in accordance with a sixth embodiment of the present invention;

FIG. 15A shows the monitor screen of an examination personal computer included in the endoscopic system of the sixth embodiment;

FIG. 15B shows the monitor screen of a portable personal computer included in the endoscopic system of the sixth embodiment;

FIG. 16 shows the configuration of an endoscope system included in a variant of the endoscopic system of the first embodiment of the present invention; and

FIG. 17 is a block diagram showing the components of an examination personal computer included in a variant of the endoscopic system of the first embodiment of the present invention.

### DETAILED DESCRIPTION OF THE PREFERRED EMBODIMENTS

Referring to the drawings, embodiments of the present invention will be described below.

To begin with, a first embodiment of the present invention will be described below.

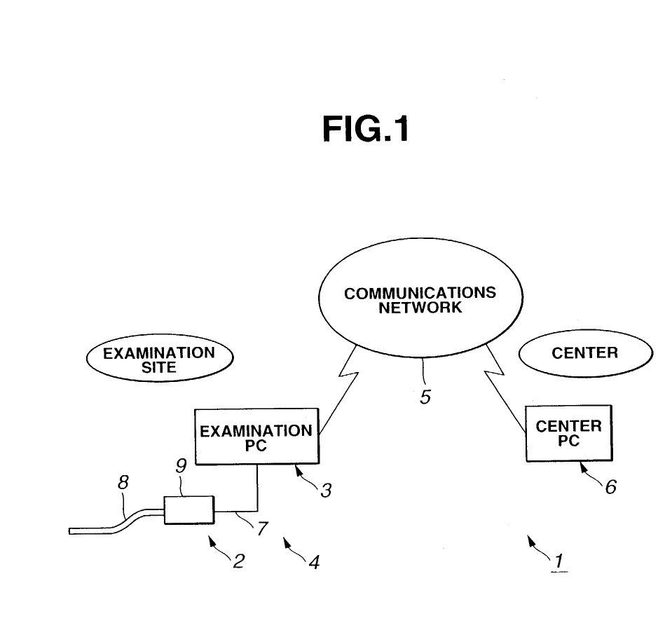

As shown in FIG. 1, an endoscopic system 1 in accordance with the first embodiment of the present invention consists mainly of: an endoscope system 4 including an endoscope 2 that is installed at an examination site at which a human being cannot stay and that is used to perform endoscopic examination, and an examination personal computer 3 (examination PC 3) that is a personal computer adopted as a general-purpose control means for controlling the endoscope 2; and, for example, a center personal computer 6 (center PC 6) that is connected to the examination PC 3 over a communications network 5 such as a LAN or the Internet and that is installed in a place far away from the examination site.

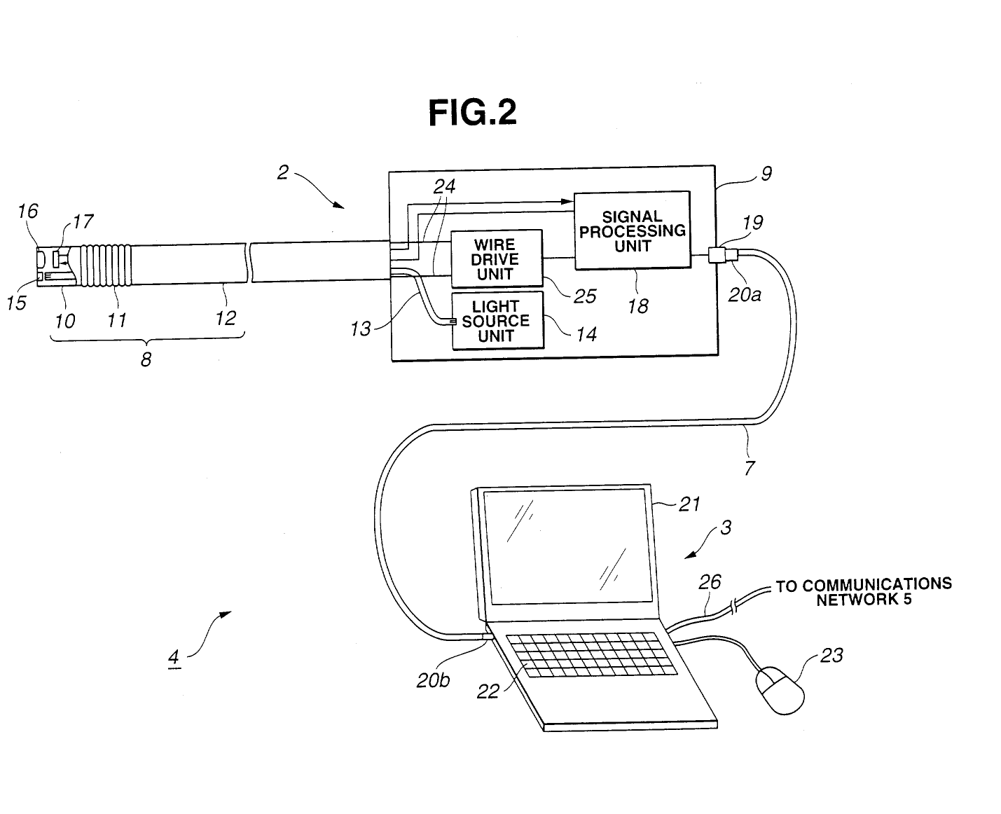

FIG. 2 shows the configuration of the endoscope system 4. The endoscope system 4 includes: the endoscope 2 used to perform endoscopic examination; and the examination PC 3 that is connected to the endoscope 2, that has the capability of a display means on which an image produced by the endoscope 2 is displayed, and that is a general-purpose control means for controlling the endoscope 2 on a centralized basis, or more particularly, for example, a notebook personal computer. The endoscope 2 and examination PC 3 are connected to each other over a single universal serial bus cable (hereinafter, referred to as USB cable) 7 over which image data produced by the endoscope 2 or an instruction issued from the examination PC 3 is transmitted.

The examination PC 3 is realized with a locally procurable personal computer but not with a personal computer designed exclusively for the present embodiment.

The endoscope 2 includes an elongated insertion member 8 that can be inserted towards a region to be examined and a control member 9 located at the rear end of the insertion member 8.

The insertion member 8 includes: a hard distal section 10 that is the distal part of the insertion member 8; a bending section 11 located at the rear end of the distal section 10 and freely bendable; and a flexible section 12 that is located at the rear end of the bending section 11 and coupled to the control member 9 and that has flexibility.

A light guide 13 over which illumination light is propagated lies through the insertion member 8, and has the rear end thereof routed to a light source unit 14 incorporated in the control member 9. The light source unit 14 has a lamp incorporated therein. Light emanating from the lamp falls on the end surface of the light guide 13, and then travels to the distal end of the light guide 13.

The distal end of the light guide 13 is locked in an illumination window included in the distal section 10. The light is emitted forwards from the distal end of the light guide 13 by way of an illumination lens 15. Consequently, an object is illuminated with the light. An observation window adjoins the illumination window. An object image is converged by an objective lens 16 locked in the observation window. An imaging device, for example, a CCD 17 is located at the position of the image plane of the objective lens. The CCD 17 photoelectrically converts the object image.

The CCD 17 is connected to a signal processing unit 18 incorporated in the control member 9 over a signal line lying through the insertion member 8. The signal processing unit 18 drives the CCD 17 and processes an output signal of the CCD 17 so as to produce a video signal. Moreover, the video signal is compressed and then converted into a signal that can be transmitted over the USB cable 7 serving as a communicating means (that supports both transmission and reception).

The output signal of the signal processing unit 18 is transmitted to the examination PC 3 over the USB cable 7 that has a USB connector 20a fixed to one end thereof and has a USB connector 20b fixed to the other end thereof. Herein, the USB connector 20a is joined with a USB connector receptacle 19, and the USB connector 20b is coupled to the examination PC 3.

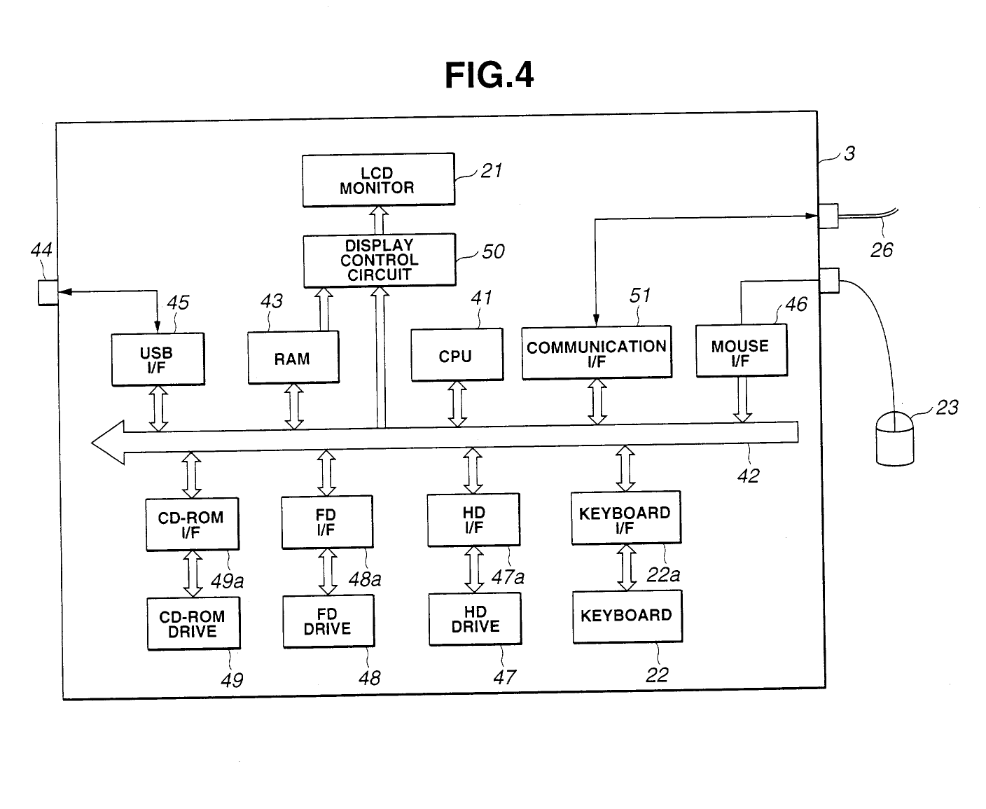

The examination PC 3 restores a signal received through the USB connector 20b to reproduce an original video signal, and displays an image converged on the CCD 17 on an LCD monitor 21. Not only an image produced by the endoscope 2 but also representations of features including a feature of remotely controlling angling of the endoscope 2 are, as described later with reference to FIG. 4, displayed on the LCD monitor 21.

The examination PC 3 includes a keyboard 22 for inputting data or the like, a mouse 23 for operating various instructions as a pointing device, and the LCD monitor 21 (hereinafter, simply, monitor) on which an endoscopic image and an endoscope control panel image containing representations of angulation buttons and others that help a user control the endoscope 2 are displayed.

Moreover, wires 24 to be used to bend the bending section 11 lie through the insertion member 8 of the endoscope 2. The rear ends of the wires 24 are coupled to a wire drive unit 25 incorporated in the control member 9. As described later, the mouse 23 is clicked in order to designate any of the angulation buttons depicted in the endoscope control panel image displayed on the LCD monitor 21. Consequently, a motor or the like incorporated in the wire drive unit 25 is driven in order to pull the wires 24. This causes the bending section 11 to bend in any of upward, downward, rightward, and leftward directions. Thus, the bending section 11 can be advanced through a tortuous lumen or the like, and the distal section 10 can be oriented in a desired direction of observation.

Moreover, the examination PC 3 is connected on the communications network 5 such as a LAN or the Internet over a cable 26.

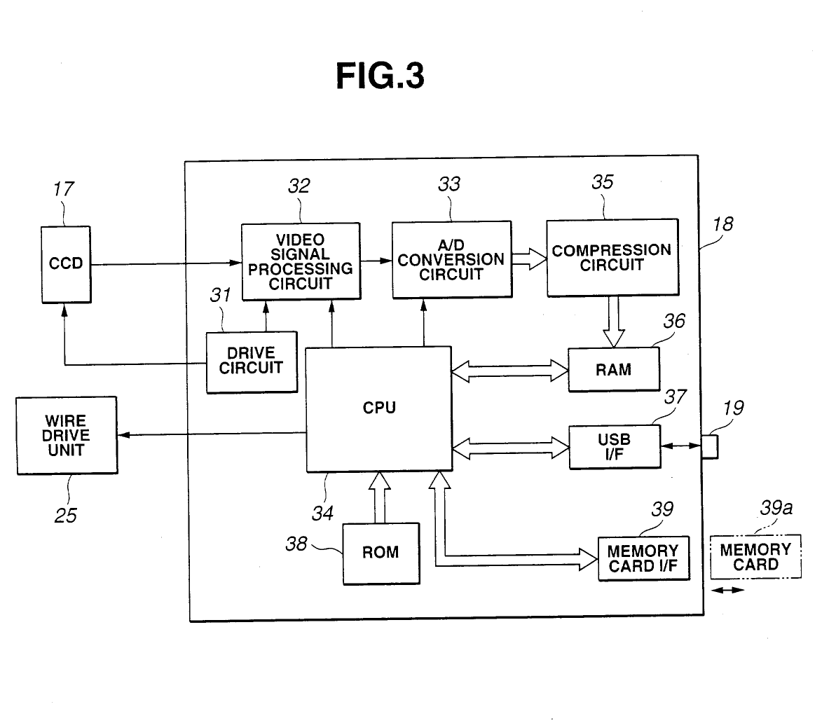

FIG. 3 shows the internal components of the signal processing unit 18.

A drive circuit 31 generates a signal with which the CCD 17 is driven and a timing signal that is applied to a video processing circuit 32. With application of a driving signal sent from the drive circuit 31, the CCD 17 transfers a signal resulting from photoelectric conversion and accumulation of charges. The output signal is transferred to the video processing circuit 32.

The video processing circuit 32 performs amplification, color separation, and others on an image signal sent from the CCD 17 so as to produce a video signal. The video signal is transferred to an A/D conversion circuit 33. A gain to be given by the video processing circuit 32 is varied in order to change the brightness of an image. Changing the brightness of an image is started with a control signal sent from a CPU 34.

The A/D conversion circuit 33 converts an output signal (analog signal) of the video processing circuit 32 into digital image data. Based on an instruction issued from the CPU 34, the A/D conversion circuit 33 can analog-to-digital convert the output signal of the video processing circuit 32 over the whole duration of the signal or can analog-to-digital convert the output signal over a partial duration thereof. In the latter case, only part of image data produced by the CCD 17 is enlarged. When an image must be displayed in enlargement, a Tele button that is not shown and that is depicted in the endoscope control panel image as described later is clicked.

The image data sent from the A/D conversion circuit 33 is transferred to a compression processing circuit 35. The compression processing circuit 35 compresses the image data according to a compressing method suitable for the received image data. The compressed image data is written (stored) in a RAM 36 serving as a memory means. The CPU 34 controls the RAM 36, and reads the image data written in the RAM 36.

The CPU 34 transfers image data in succession to a USB interface (USB I/F in FIG. 2 and others) 37. The CPU 34 operates according to any of programs stored in a ROM 38. A memory card interface 39 is connected to the CPU 34. Image data may be recorded in a memory card 39a that is detachably attached to the memory card interface 39.

The image data recorded in the memory card 39a is read by the CPU 34 later, and transferred to the USB interface 37.

A lid that is structured to keep a memory card chamber watertight and that is not shown is opened in order to remove the memory card 39a or replace the memory card 39a with another. A user can select the memory card 39a of an appropriate storage capacity and loaded in the chamber.

Moreover, the USB interface 37 converts the image data into a signal conformable to the USB standard. The USB interface 37 is connected to the USB connector receptacle 19. The image data is transmitted to the examination PC 3 over the USB cable 7 that has the USB connector 20a thereof joined to the USB connector receptacle 19.

The foregoing actions are repeatedly performed at intervals of a frame time in television, that is, a one-thirtieth (NTST system) or one-twenty fifth (PAL system) of one sec. The signal processing unit 18 is composed of semiconductor devices and is therefore very small in size. This means that the control member 9 of the endoscope 2 shown in FIG. 2 is small in size. Moreover, since an operating means is unnecessary, the endoscope 2 can be placed in any place out of human beings' reach.

FIG. 4 shows the internal components of the examination PC 3. The examination PC 3 having the components is not designed exclusively for the present embodiment but is a general-purpose examination PC locally procurable nowadays. A USB interface 45 and a USB connector receptacle 44 are included in almost all models of examination PCs locally procurable nowadays.

The examination PC 3 has a CPU 41 thereof, which controls all the components, connected on an internal bus 42. A RAM 43 used as a work area of the CPU 41, the USB interface 45 connected to the USB connector receptacle 44, and a mouse interface 46 to which a connector fixed to a cord extending from the mouse 23 is connected (via a connector receptacle) are interconnected over the internal bus 42.

Moreover, the keyboard 22, a hard disk (HD in the drawing) drive 47 in which programs are saved, a flexible disk (referred to as FD) drive 48 for a floppy (floppy is a trademark) disk or the like, and a CD-ROM drive 49 are interconnected over the internal bus 42 via interfaces 22a, 47a, 48a, and 49a respectively. Moreover, a communication interface 51 is also connected on the internal bus 42. The communication interface 51 is connected on the communications network 5 such as a LAN or the Internet over the cable 26.

The LCD monitor 21 is connected to the RAM 43 and on the internal bus 42 via a display control circuit 50 that controls display.

The CPU 41 reads a program stored in the hard disk drive 47, writes it in a predetermined area within the RAM 43, and acts based on the program. The program is described in order to display a predetermined screen image on the monitor 21 of the examination PC 3.

The CPU 41 prepares screen display data in the predetermined area within the RAM 43. The display control circuit 50 repeatedly reads the screen display data and converts it into a signal, based on which a screen image is displayed on the monitor 21, all the time. The signal is sent to the monitor 21, and the screen image is displayed based on the signal.

Image data sent from the endoscope 2 to the examination PC 3 is received through the USB interface 45, transferred to over the internal bus 42, and read by the CPU 41. According to a program, the CPU 41 restores the image data to its original uncompressed image data, and writes it in the predetermined area in the RAM 43 so that an image will be displayed on the monitor 21. This action is repeatedly performed at intervals of the frame time in television, that is, a one-thirtieth sec (to be exact, 1/29.94 sec) or a one-twenty fifth sec.

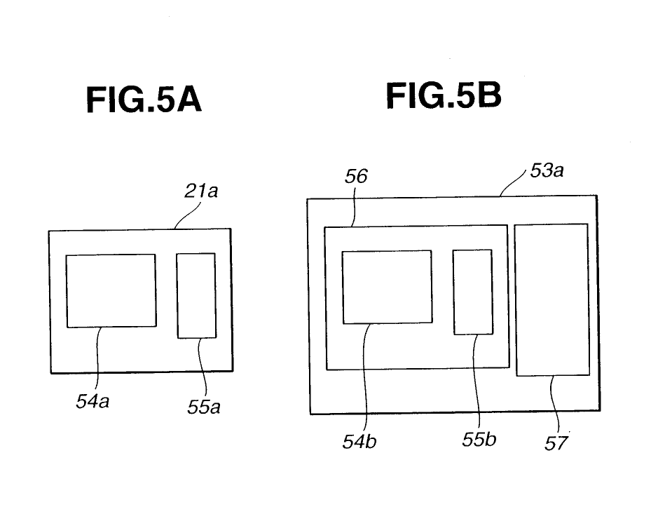

Moreover, an endoscopic image 54a converged on the CCD 17 included in the endoscope 2 and the endoscope control panel image 55a helping a user operate the endoscope 2, for example, control angulation of the endoscope are, as shown in FIG. 5A, displayed on a monitor screen 21a of the monitor 21.

The keyboard 22 or mouse 23 is handled in order to move an operating cursor to a desired one of various buttons depicted in the endoscope control panel image 55a. The mouse 23 is then clicked on the desired button, whereby a feature associated with the button works.

The endoscope control panel image 55a contains the representations of the angulation buttons and others. By manipulating the angulation buttons, or to be exact, by manipulating the representations of the angulation buttons, the bending section 11 of the endoscope 2 can be bent in any desired direction.

In the endoscope system 4 included in the present embodiment, the endoscope 2 and examination PC 3 are connected to each other over the USB cable 7. The endoscope 2 is controlled based on a control instruction sent from the examination PC 3 over the USB cable 7. Image data produced by the endoscope 2 is transmitted to the examination PC 3 over the USB cable 7, whereby an image represented by the image data is displayed on the monitor 21. Owing to the foregoing components, the examination PC 3 may be realized with any of locally procurable PCs serving as centralized controllers. A dedicated centralized controller is unnecessary. Consequently, the endoscope system 4 can be realized at low cost.

Moreover, since a notebook PC can be adopted as the examination PC 3, the examination PC 3 is compact and lightweight and easy to carry.

If the examination PC 3 fails, any of various types of examination PCs can be substituted for the examination PC 3 in order to control the endoscope 2 or display an image. Consequently, endoscopic examination can be performed uninterruptedly. Moreover, since the examination PC 3 has the capability of a centralized controller, any other computer need not be prepared in order to preserve view images produced by the endoscope 2.

The other center PC 6 has the same components as, for example, the examination PC 3. The description of the components will therefore be omitted. Needless to say, a desktop personal computer may be adopted as the center PC 6 or examination PC 3.

In the endoscopic system 1 of the present embodiment, existing communications software, for example, NetMeeting developed by Microsoft Corp. is installed in both the PCs 3 and 6 so that data can be transferred or communicated between the PCs 3 and 6.

To be more specific, according to the present embodiment, the same communications software is installed in both the PCs 3 and 6. A copy of contents to be displayed on the monitor 21 of the examination PC 3 is displayed on a monitor screen 53a of the monitor of the center PC 6. An examination PC window 56 is, as shown in FIG. 5B, opened, that is, displayed on the monitor screen 53a.

Information provided by the examination PC 3 can also be acquired through the examination PC window 56. If an operating means is displayed, manipulations can be performed using the operating means in the same manner as they are using the examination PC. More particularly, an endoscopic image 54b similar to the endoscopic image 54a and an endoscope control panel image 55b similar to the endoscope control panel image 55a are displayed within the examination PC window 56. The endoscopic image 54b can be checked as if an endoscopic image produced by the endoscope 2 were checked using the examination PC 3. Moreover, when manipulations are performed through the endoscope control panel image 55b, an instructing signal produced responsively is transmitted to the CPU 41 in the examination PC 3 over the communications network 5. The CPU 41 extends control as if the manipulations were performed through the endoscope control panel image 55a on the monitor screen 21a.

According to the present embodiment, a window 57 dedicated to the run of another software, for example, image management software for managing images is opened on the monitor screen 53a. Endoscopic images and relevant information which result from endoscopic examination can be preserved or managed through the window 57.

Next, an operation to be exerted by the present embodiment will be described below.

As shown in FIG. 5A, the endoscopic image 54a produced by the endoscope 2 and the control panel image 55a helping a user operate the endoscope 2, for example, control angulation of the endoscope 2 are displayed on the monitor screen 21a of the monitor 21 of the examination PC. A user performs manipulations through the control panel image 55a so as to given control or angle the endoscope 2.

As shown in FIG. 5B, the examination PC window 56 containing the same contents as those displayed on the monitor 21 of the examination PC 3 is opened on the monitor screen 53a of the monitor of the center PC 6.

Within the examination window 56, the endoscopic image 54b produced by the endoscope 2 and the control panel image 55b assisting in operating the endoscope 2, for example, angling the endoscope are displayed.

Through the examination PC window 56 opened on the center PC 6, manipulations can be performed through software running in the examination PC 3.

For example, manipulations are performed through the endoscope control panel image 55b displayed within the examination PC window 56 opened on the center PC 6, whereby image data produced by the endoscope 2 may be transferred to the examination PC 3 or the endoscope 2 itself can be operated.

Moreover, image management software is installed in the center PC 6, and manipulations are performed through the window 57 dedicated to the run of the image management software. Thus, endoscopic image data preserved in the examination PC 3 may be transferred to the center PC 6 and managed comprehensively by the center PC 6.

As mentioned above, in the endoscopic system 1 of the present embodiment, the examination PC 3 is used to control or operate the endoscope 2. Moreover, the center PC 6 connected to the examination PC 3 over the communications network 5 may be used to control or operate the endoscope 2 in the same manner as the examination PC 3 is used.

In other words, since the endoscopic image 54b and endoscope control panel image 55b that are identical to those displayed on the monitor screen 21a of the examination PC 3 are displayed on the monitor screen 53a of the center PC 6. Consequently, the endoscope 2 can be controlled in the same manner as it is directly controlled using the monitor screen 21a of the examination PC 3.

Consequently, even if a user cannot directly operate the endoscope 2 because the endoscope 2 lies in a remote place far away from the user or in a radioactively contaminated region, the same contents as those displayed on the monitor 21 of the examination PC 3, that is, the endoscopic image 54b and endoscope control panel image 55b are displayed within the examination PC window 56 on the center PC 6. Therefore, a user can control the endoscope 2 using the center PC 6 disposed in a remote place as if the user actually controlled the endoscope system 4, and can then perform endoscopic examination.

Moreover, a user can preserve or manage the produced endoscopic image 54b while staying in a remote place.

Referring to FIG. 1, a wireless LAN may be adopted as the communications network 5 such as a LAN or the Internet.

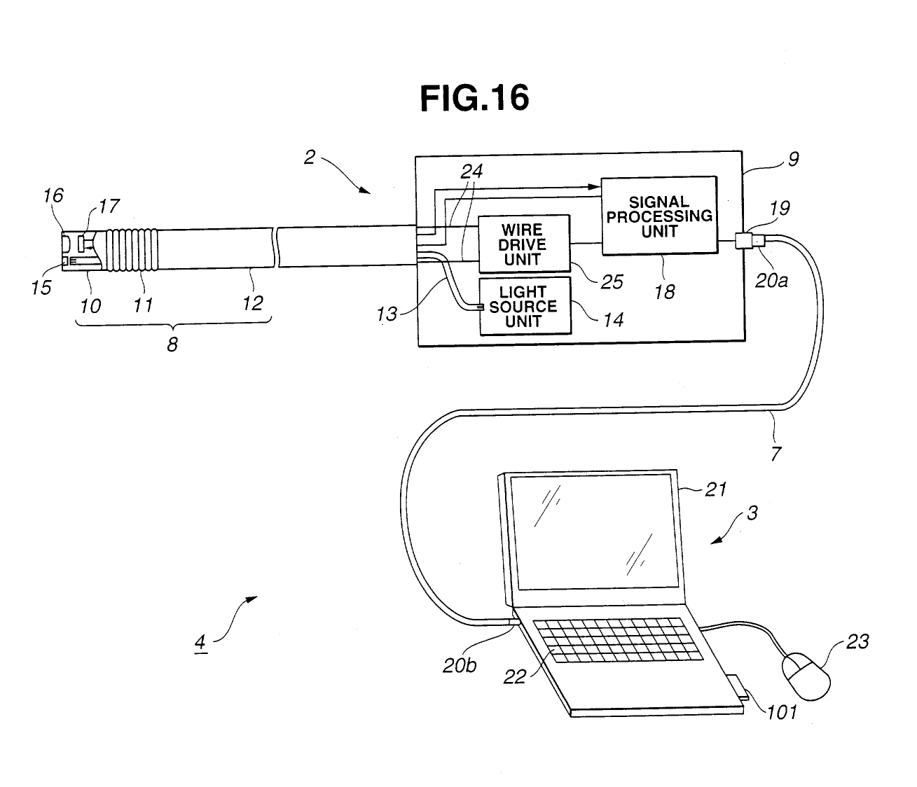

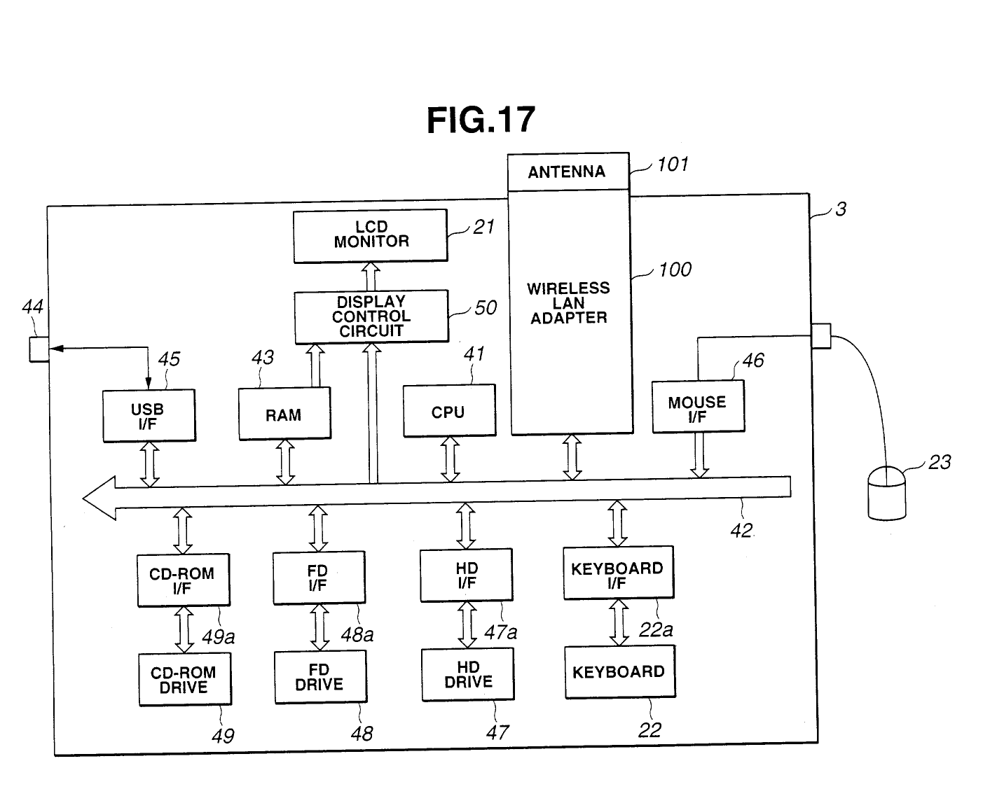

FIG. 16 shows a variant of the first embodiment, showing the configuration of an endoscopic system using a wireless LAN instead of the Internet included in the endoscopic system of the first embodiment. FIG. 17 is a block diagram showing the components of an examination PC.

Referring to FIG. 17, a wireless LAN adaptor 100 is substituted for the communication interface 51 shown in FIG. 4 and connected on the internal bus 42. The wireless LAN adaptor 100 includes an antenna 101 as a distal part thereof. The wireless LAN adaptor 100 is, for example, model WLI-PCM-L11G produced by Melco Inc.

Referring to FIG. 16, the antenna 101 included in the wireless LAN adaptor is exposed from the examination PC 3 instead of the cable 26 shown in FIG. 2.

When the wireless LAN adaptor is included in both the examination PC 3 and center PC 6 shown in FIG. 1, the PCs are connected to each other over the communications network by a wireless LAN.

The present embodiment provides the advantages described below.

The endoscope can be operated using the center PC 6, which is located in a place far away from the examination PC 3, as if it were directly operated using the examination PC 3. This leads to improved maneuverability.

If the examination endoscope system 4 is placed at an examination site that is a remote place, a radioactively contaminated region, or any other place in which a human being cannot stay and operate an endoscope, the center PC 6 disposed in a place away from the examination site can be used to carry out endoscopic examination while enjoying excellent maneuverability. Moreover, acquired image data can be preserved and managed.

Next, a second embodiment of the present invention will be described below.

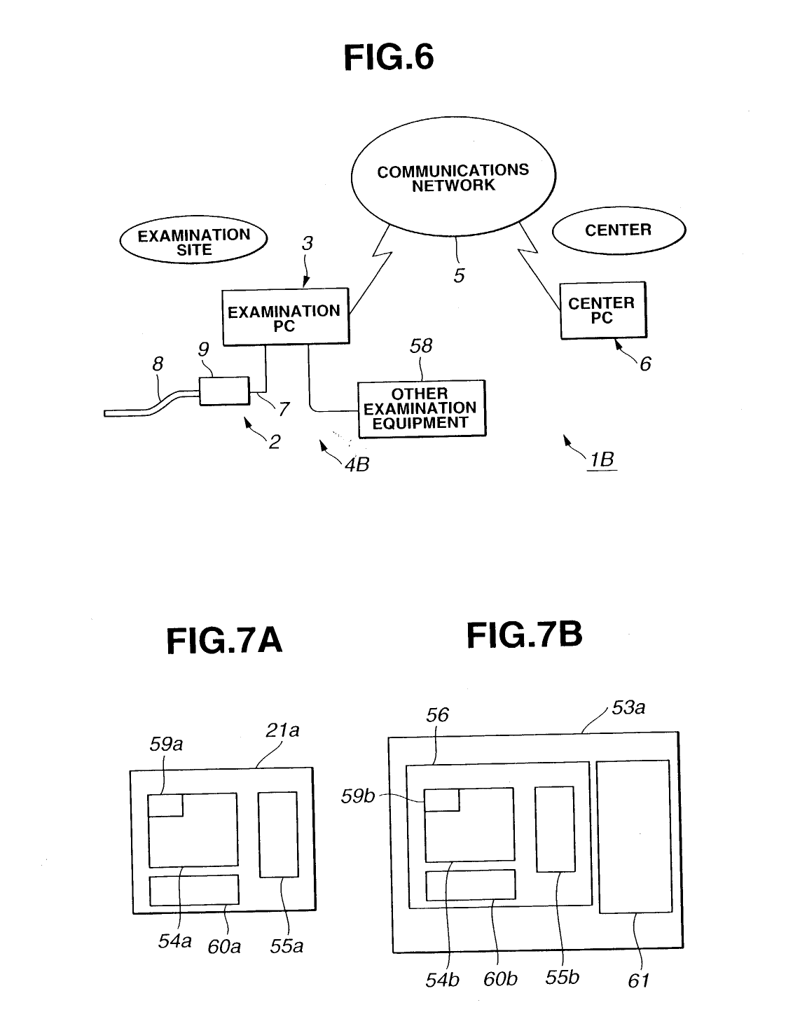

FIG. 6 shows an endoscopic system 1B in accordance with the second embodiment of the present invention.

In the endoscopic system 1B, an endoscope system 4B includes, in addition to the same components as those of the endoscope system 4 shown in FIG. 1, other examination equipment 58 such as an ultrasonic examination system or an eddy current examination system that is connected to the examination PC 3.

FIG. 7A shows an example of display on the monitor screen 21a of the monitor 21 of the examination PC 3. In addition to the information shown in FIG. 5A, examination information 59a provided by the other examination equipment 58 and a control panel image 60a through which the other examination equipment 58 is operated are displayed on the monitor screen 21a.

The actions of the other examination equipment 58 can be controlled by performing manipulations through the control panel image 60a.

Moreover, as shown in FIG. 7B, similarly to FIG. 5B, the examination PC window 56 is opened on the monitor 53a of the center PC 6. A copy of the contents of display shown in FIG. 7A is displayed within the examination PC window 56.

Specifically, in addition to the information displayed within the examination PC window 56 shown in FIG. 5B, examination information 59b provided by the other examination equipment 58 and a control panel image 60b through which the other examination equipment 58 is operated are displayed.

The actions of the other examination equipment 58 can be controlled over the communications network 5 by performing manipulations through the control panel image 60b.

According to the second embodiment, the other examination equipment 58 such as an ultrasonic examination system or an eddy current examination system can be connected to the examination PC 3, and operated by handling the center PC 6 for examination.

In this case, the control panel image 60a through which the other examination equipment 58 is controlled is displayed on the monitor screen 21a of the examination PC 3. The examination information 59a provided by the other examination equipment 58 is bonded to, that is, superimposed on, for example, a corner of the endoscopic image 54a. A copy of the endoscopic image having the examination information superimposed thereon is displayed on the monitor screen 53a of the center PC 6. The endoscope can be operated using the center PC 6 as if it were operated using the examination PC 3. The examination information 59b can be preserved together with the endoscopic image 54b.

Next, a third embodiment of the present invention will be described below.

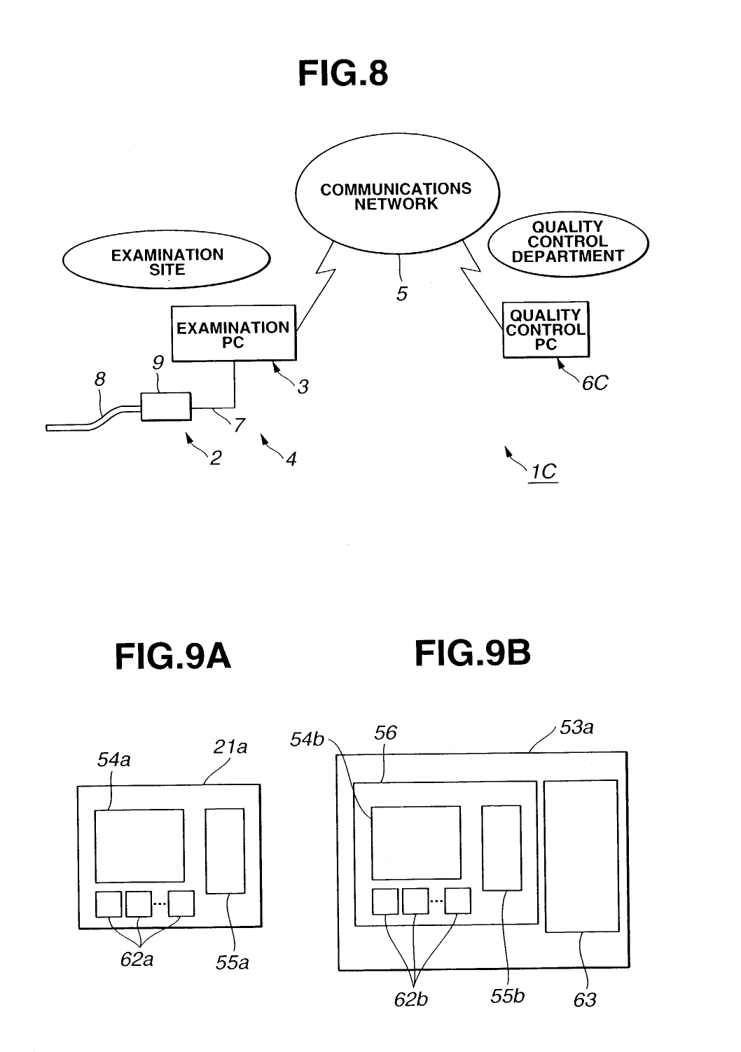

FIG. 8 shows an endoscopic system IC in accordance with the third embodiment of the present invention. The endoscopic system IC is a system adapted to quality control. The endoscopic system 1C shown in FIG. 8 has the endoscope system 4 identical to the one shown in FIG. 1 installed, for example, at an on-site examination department of a customer and a management PC 6C installed at a quality control department thereof. The management PC 6C is realized with the same computer as the one adopted as the examination PC 3. Any PC that has already been employed can be adopted as the management PC 6C.

Existing communications software is installed in both the PCs 3 and 6C so that they can be activated any time. The PCs 3 and 6C can communicate with each other over the communications network 5 such as a LAN or the Internet.

Normally, the examination PC 3 included in the endoscope system 4 installed at the on-site examination department is used to perform endoscopic examination.

The on-site examination department may have to discuss a situation with the quality control department or the quality control department may have to grasp the progress of an examination.

In such a case, the communications software installed in the PCs 3 and 6C is activated.

When the endoscope system 4 installed at the on-site examination department is used to perform endoscopic examination, what is displayed on the monitor screen 21a is as shown in FIG. 9A.

In addition to the endoscopic image 54a and endoscope control panel image 55a, index images (thumbnail images) 62a of past endoscopic images are displayed on the monitor screen 21a of the monitor 21 of the examination PC 3.

When the communications software is activated, the examination PC window 56 opens, that is, appears on the monitor screen 53a of the quality control PC 6C. A copy of the information displayed on the monitor screen 21a, or more particularly, the endoscopic image 54b, endoscope control panel image 55b, and index images (thumbnail images) 62b of the past endoscopic images are displayed within the quality control PC window 56.

Moreover, a window 63 dedicated to the run of a browser is opened on the monitor screen 53a. By performing manipulations through the window 63, a database created in the past by recording image data is accessed in order to retrieve or display any image or manage images.

Images preserved using the examination PC 3 in the onsite examination department can be transferred to the quality control PC 6C at the quality control department. The quality control department can review the images in details.

Moreover, if necessary, the quality control department remotely handles the examination PC 3 installed at the onsite examination department. Thus, past examination images preserved in the examination PC 3 may be transferred to the management PC 6C and reviewed in detail. (Manipulations are performed on basic features whose representations are contained in the examination PC window 56 opened on the monitor screen 53a of the management PC 6C.) Moreover, if necessary, the quality control department uses the quality control PC 6C to control or operate the endoscope system 4 installed at the on-site examination department. (Manipulations are performed through the endoscope control panel image 55b displayed within the examination PC window 56 opened on the monitor screen 53a of the quality control PC 6C). Thus, a staff member of the quality control department can perform endoscopic examination on behalf of the one of the on-site examination department.

Next, a fourth embodiment of the present invention will be described below.

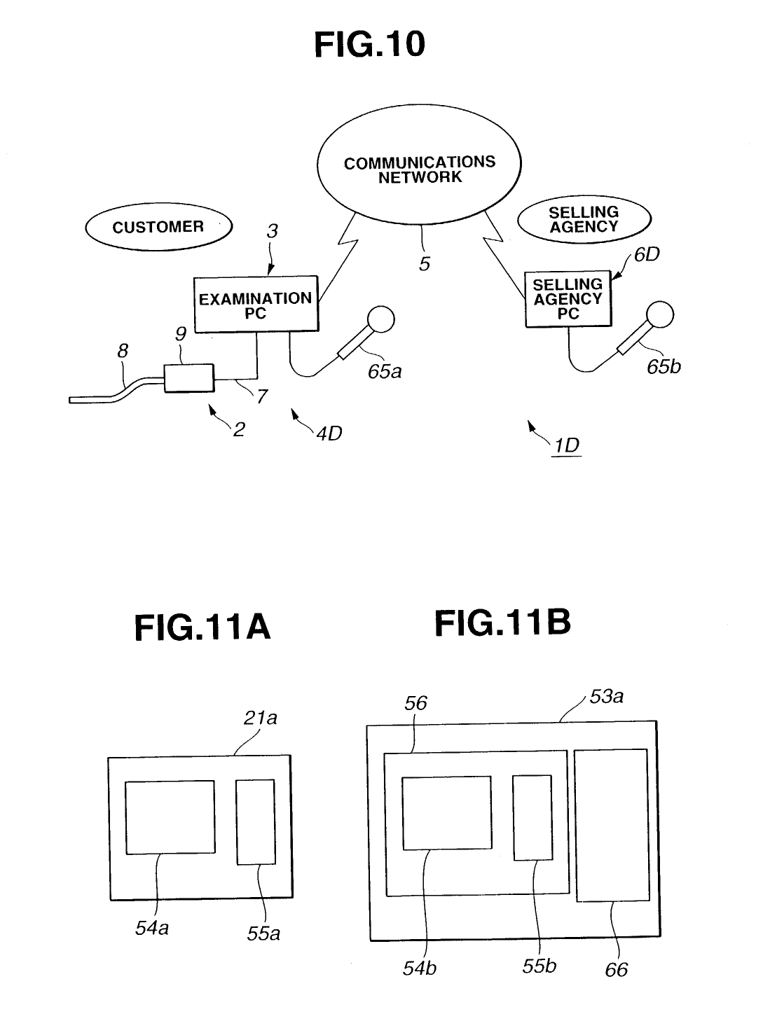

FIG. 10 shows an endoscopic system 1D in accordance with the fourth embodiment. The endoscopic system 1D of the present embodiment is suitable for training or provision of customer services.

The endoscopic system 1D includes an endoscope system 4D installed at a customer and a selling agency PC 6D that is connected to the endoscope system 4D over the communications network 5 such as a private network or the Internet and that is installed at a selling agency. The same PC as the one adopted as the examination PC 3 can be used as the selling agency PC 6D.

The endoscope system 4D includes, in addition to the same components as those of the endoscope system 4 shown in FIG. 1, a microphone 65a which is connected to the examination PC 3 and through which voice is received. Moreover, a microphone 65b is connected to the selling agency PC 6D, and the examination PC 3 and selling agency PC 6D each have a loudspeaker incorporated therein.

Existing communications software is installed in both the PCs 3 and 6D. When the communications software is activated, the PCs 3 and 6D can communicate with each other over the communications network 5.

In this case, as shown in FIG. 11A, similarly to FIG. 5A, the endoscopic image 54a and endoscope control panel image 55a are displayed on the monitor screen 21a of the monitor 21 of the examination PC 3.

Moreover, as shown in FIG. 11B, the examination PC window 56 is opened on the monitor screen 53a of the selling agency PC 6D. The endoscopic image 54b and endoscope control panel image 55b are, similarly to those shown in FIG. 5B, displayed within the examination PC window 56.

Moreover, a window dedicated to the run of other software, or more particularly, a window 66 dedicated to the run of training manual software is opened by the right side of the examination PC window 56.

Owing to the foregoing configuration, the customer can perform endoscopic examination using the endoscope system 4D. By the way, if the customer is unfamiliar with the operation of the endoscope system 4D, the customer may feel at a loss what to do.

In such a case, the customer and selling agency can communicate speech to each other using the PCs 3 and 6D respectively over the communications network 5. For example, the customer may want to ask a question about the operation of the endoscope system 4D. In this case, the customer can talk to the selling agency while looking at the monitor screen 21a. On the other hand, the selling agency can talk to the customer and given an appropriate advice thereto while looking at the examination PC window 56 on the monitor screen 53a of the selling agency PC 6D.

Although the customer is given the advice, the customer may not be able to understand well. In this case, the selling agency handles the selling agency PC 6D so as to operate the endoscope system 4D on behalf of the customer. (For example, the selling agency performs manipulations through the endoscope control panel image 55b displayed within the examination PC window 56 on the monitor screen 53a of the selling agency PC 6D.)

Moreover, the selling agency performs manipulations through the window 66 opened on the monitor screen 53a so as to reference a training manual that gives an easy-to-understand answer to the customer's question. Thus, the selling agency can give an easy-to-understand advice to the customer.

As mentioned above, the same screen image as the one displayed on the monitor screen 21a of the customer's examination PC 3 can be seen using the selling agency PC 6D located in a place far away from the endoscope system 4D installed at the customer. The customer and selling agency can communicate speech to each other using both the PCs 3 and 6D. Furthermore, the endoscope can be operated using the selling agency PC 6D as if it were directly operated using the customer's examination PC 3. The selling agency can provide the customer, who is unfamiliar with the operation of the endoscope system 4D, with training or customer services from a place far away from the customer.

According to the present embodiment, the selling agency can effectively provide the customer with training or services relevant to the endoscope system 4D they have sold to the customer.

Next, a fifth embodiment of the present invention will be described below.

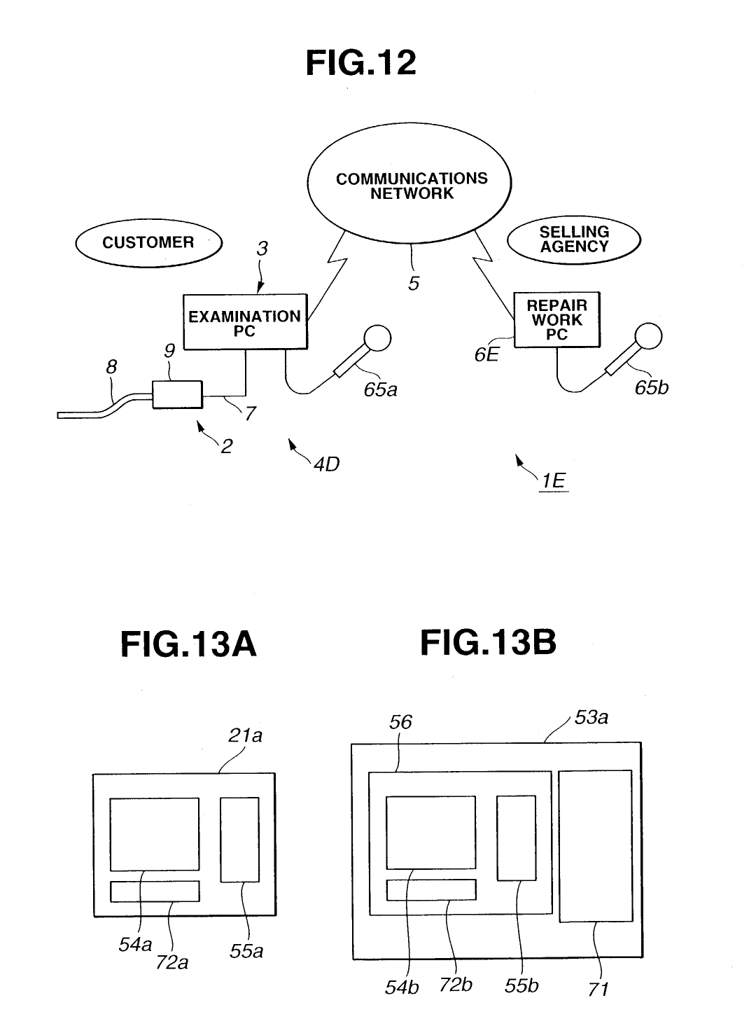

FIG. 12 shows an endoscopic system 1E in accordance with the fifth embodiment of the present invention. The present embodiment is an endoscopic system suitable for remotely controlling repair or evaluation.

The endoscope system 1E includes a repair work PC, that is, a repair work PC 6E instead of the selling agency PC 6D included in the endoscopic system 1D shown in FIG. 10.

To be more specific, the endoscope system 4D is installed at a customer, and the examination PC 3 included in the endoscope system 4D is connected to the repair work PC 6E installed at a selling agency over the communications network 5.

Existing communications software is installed in both the PCs 3 and 6E. When the communications software is activated, the PCs 3 and 6E can communicate with each other over the communications network 5 such as a private network or the Internet.

The customer can perform endoscopic examination using the endoscope system 4D. In this case, as shown in FIG. 13A, the same information as the one shown in FIG. 5A is displayed on the monitor screen 21a of the monitor 21.

Moreover, as shown in FIG. 13B, the examination PC window 56 whose contents are identical to those displayed on the monitor screen 21a is opened on the monitor screen 53a of the repair work monitor 6E.

According to the present embodiment, other window, for example, a window 71 dedicated to the run of repair manual software is opened on the monitor screen 53a. When manipulations are performed through the window 71, control data can be transmitted from the repair work PC 6E to the examination PC 3 over the communications network 5. The CPU 41 included in the examination PC 3 checks the current state of the endoscope system 4D including the controlled state of the endoscope 2 and the controlling state of the examination PC 3, and indicates the states using a state indicator panel image 72a displayed on the monitor screen 21a.

When the state indicator panel image 72a is displayed on the monitor screen 21a, a state indicator panel image 72b identical to the state indicator panel image 72a is displayed within the examination PC window 56 on the monitor screen 53a.

The state indicator panel image 72a and state indicator panel image 72b assist in evaluation required in order to judge whether repair must be performed.

An operation to be exerted by the foregoing system configuration will be described below.

If any defect occurs in the customer's endoscope system 4D, the customer activates the communications software installed in the examination PC 3 so that the examination PC 3 can communicate with the repair work PC 6E at the selling agency over the communications network 5 such as a private network or the Internet.

Microphones 65a and 65b are connected to the customer's examination PC 3 and repair work PC 6E respectively. Moreover, a loudspeaker is incorporated in each of the examination PC 3 and repair work PC 6E. Therefore, the customer and selling agency can communicate speech to each other by way of the PCs 3 and 6E.

As shown in FIG. 13A, the endoscopic image 54a and endoscope control panel image 55a are displayed on the monitor screen 21a of the customer's examination PC 3. The same screen image as the one displayed on the customer's examination PC 3 is displayed within the examination PC window 56 on the monitor screen 53a of the repair work PC 6E installed at the selling agency. While the selling agency is talking to the customer at the sight of the window 56, the selling agency learns what kind of defect has occurred in the customer's endoscope system 4D.

If the selling agency cannot still fully understand the nature of the defect, the selling agency handles the repair work PC 6E so as to operate the endoscope system 4D on behalf of the customer. (The selling agency performs manipulations through the endoscope control panel image 55b displayed within the examination PC window 56 on the monitor screen 53a of the repair work PC 6E.)

If necessary, manipulations may be performed through the window 71 that is dedicated to the run of repair manual software and that is opened on the repair work PC 6E. Thus, the state of the endoscope system 4D may be displayed on the monitor screen 21a and within the examination PC window 56 respectively. A staff member at the selling agency may make a judgment on the state. If necessary, the repair manual software may be referenced in order to proceed with evaluation smoothly.

As mentioned above, the repair work PC 6E installed at the selling agency located away from the endoscope system 4D for examination installed at the customer is used to see the same screen image as the one displayed on the examination PC 3 at the customer. By handling the repair work PC 6E the endoscope can be operated as if it were directly operated by handling the customer's examination PC 3. The selling agency need not visit the customer, and the customer need not deliver the endoscope system 4D back to the selling agency. Nevertheless, the selling agency can judge what defect has occurred in the customer's endoscope system 4D, and therefore can carry out repair work immediately.

Next, a sixth embodiment of the present invention will be described below.

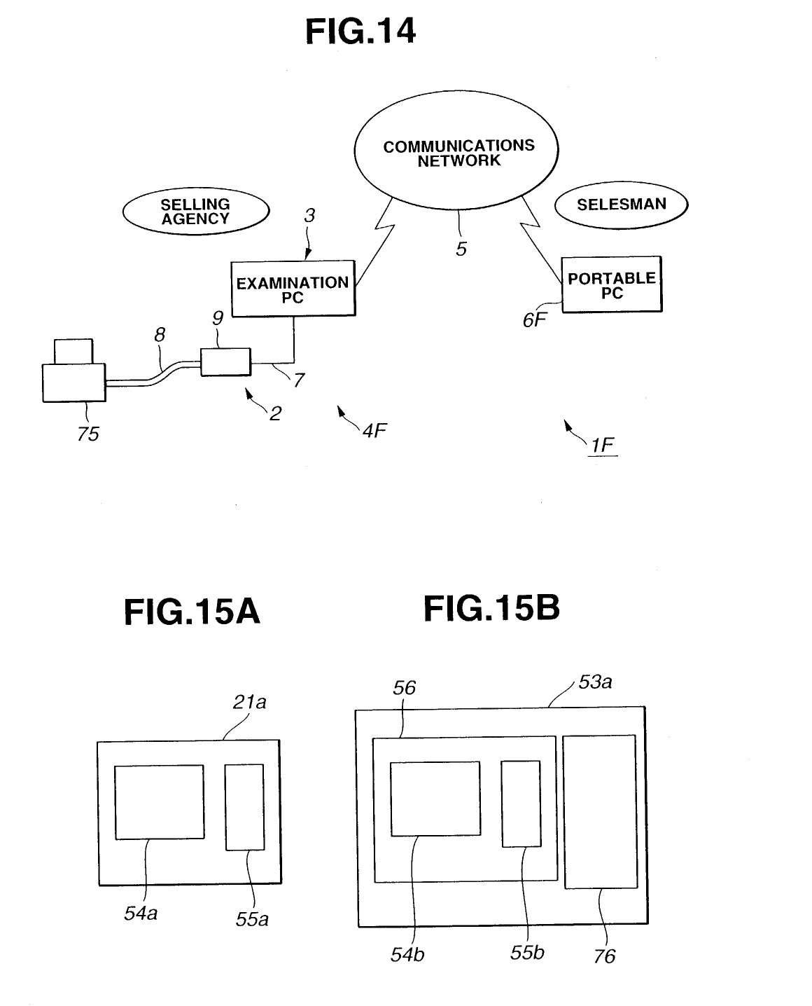

FIG. 14 shows an endoscopic system IF in accordance with the sixth embodiment of the present invention. The present embodiment is an endoscopic system suitable for, for example, demonstration that is performed as a sales promotion. The endoscopic system IF consists mainly of an endoscope system 4F installed at a selling agency, and a portable PC 6F that is connected to the examination PC 3 included in the endoscope system 4F over the communications network 5 such as a private network or the Internet and that is designed to be carried by, for example, a salesperson. In this case, the endoscope system 4F includes, in addition to the same components as those of the endoscope system 4 shown in FIG. 1, an object of examination 75 such as an engine, a boiler, or a model of a human body cavity. The endoscope 2 is used to perform endoscopic examination on the object of examination 75.

Incidentally, the portable PC 6F may be identical to the examination PC 3 or may be a mobile personal computer that is more compact and lightweight.

A salesperson visits a customer while carrying the portable PC 6E alone.

Existing communications software is installed in both the PCs 3 and 6F. When the communications software is activated, the PCs 3 and 6F can communicate with each other over the communications network 5.

As shown in FIG. 15A, the endoscopic image 54a and endoscope control panel image 55a are displayed on the monitor screen 21a of the examination PC 3 installed at the selling agency.

As shown in FIG. 15B, the same information as the one displayed on the monitor screen 21a of the examination PC 3 installed at the selling agency, that is, the endoscopic image 54b and endoscope control panel image 55b are displayed within one examination PC window 56 opened on the monitor screen 53a of the portable PC 6F.

Moreover, a window 76 dedicated to the run of software that displays a publicity catalogue or publicity video is opened on the monitor screen 53a. Features of the endoscope system 4F are graphically introduced by performing manipulations through the window 76.

Next, an operation to be exerted by the present embodiment will be described below.

A salesperson handles the portable PC 6F, or more particularly, performs manipulations through the endoscope control panel image 55b within the examination PC window 56 on the monitor screen 53a of the portable PC 6F. Thus, the salesperson can perform manipulations as if he/she directly performed manipulations on the examination PC 3. The salesperson need not carry the endoscope system 4F to a customer but need carry only a catalogue and the portable PC 6F to visit the customer. The salesperson can now demonstrate a scene of endoscopic examination to be performed using the endoscope system 4F. Moreover, if the salesperson allows the customer to perform manipulations through the endoscope control panel image 55b, the customer can experience endoscopic examination.

Consequently, the salesperson need not carry the endoscope system 4F to a customer but should merely explain the features of the system so that the customer can fully understand them. Thus, the salesperson can carry out sales activities, or in other words, sales activities can be carried out readily.

According to the first embodiment, the examination PC window 56 is opened on the monitor screen 53a of the center PC 6 and a copy of the information displayed on the examination PC 3 is displayed within the examination PC window 56. Alternatively, the endoscopic image 54a and endoscope control panel image 55a may be displayed on the monitor screen 53a with the display sizes thereof changed. Moreover, the examination PC 3 and others are not limited to existing personal computers but may be any computers as long as the computers include a CPU. Incidentally, a combination of parts of the aforesaid embodiments also belongs to the present invention.

As mentioned above, according to the present invention, an endoscope can be controlled using a second personal computer as if it were controlled using a first personal computer.

In the present invention, it is apparent that a wide range of different working modes can be formed based on the invention without a departure from the spirit and scope of the invention. The present invention is not restricted to any specific embodiment but is limited to the appended claims.

What is claimed is:

1. An endoscopic system comprising:
an endoscope to be used to perform endoscopic examination;
a first personal computer having a first display unit on which an endoscopic image produced by said endoscope and a control panel image through which said endoscope is controlled or operated are displayed; and
a second personal computer connected to said first personal computer over a communications network, wherein:
the same endoscopic image and control panel image as those displayed on said first display unit are displayed on a second display unit included in said second personal computer; and
said endoscope can also be controlled or operated through the control panel image displayed on said second display unit.

2. An endoscopic system according to claim 1, wherein an operating window can be opened on said second display unit.

3. An endoscopic system according to claim 2, wherein said operating window is dedicated to the run of image browser software.

4. An endoscopic system according to claim 2, wherein said operating window is dedicated to the run of repair manual software.

5. An endoscopic system according to claim 1, further comprising examination equipment.

6. An endoscopic system according to claim 5, wherein an operating window can be opened on said second display unit.

7. An endoscopic system according to claim 6, wherein said operating window is an operating window through which said examination equipment is operated.

8. An endoscopic system according to claim 1, wherein index images of endoscopic images can be displayed on said first and second display units.

9. An endoscopic system according to claim 1, wherein said first and second personal computers can be operated with the same kind of communications software installed therein.

10. An endoscopic system according to claim 1, wherein endoscopic images preserved in said first personal computer can be comprehensively managed using said second personal computer.

11. An endoscopic system according to claim 1, wherein said communications network is a wireless LAN.

12. An endoscopic system comprising:
an endoscope to be used to perform endoscopic examination;
a first controller having a first display unit on which an endoscopic image produced by said endoscope and a control panel image through which said endoscope is controlled or operated are displayed; and
a second controller connected to said first controller over a communications network, wherein:
the same endoscopic image and control panel image as those displayed on said first display unit are displayed on a second display unit included in said second controller; and
said endoscope can be controlled or operated through the control panel image displayed on said second display unit.

13. An endoscopic system according to claim 12, wherein said first and second controllers are realized with personal computers.
